(19) United States
(12) Patent Application Publication
(10) Pub. No.: US 2015/0007827 A1
(43) Pub. Date: Jan. 8, 2015

(54) OCCLUSION DEVICE WITH OPENABLE CHANNEL

(71) Applicant: Conceptus, Inc., Mountain View, CA (US)

(72) Inventors: Feridun Ozdil, Danville, CA (US); Christopher A. Stout, San Bruno, CA (US)

(21) Appl. No.: 13/933,982

(22) Filed: Jul. 2, 2013

(51) Int. Cl. A61F 6/22; A61B 19/00

(52) U.S. Cl. CPC A61F 6/225; A61B 19/54; A61B 2019/5466
USPC 128/831

(57) ABSTRACT

The various embodiments of the present invention provide implantable occlusion devices and methods of using the occlusion devices. In an embodiment, the occlusion devices include an openable channel to facilitate reversible female contraception.

### BACKGROUND OF THE INVENTION

1. Field of the Invention

Embodiments of the present invention relate to the field of minimally invasive surgical medical devices and medical procedures. More specifically, embodiments of the present invention relate to devices and methods used for transcervical gynecological procedures.

2. Discussion of Related Art

Female contraception and sterilization may be enabled by transcervically introduced fallopian tube inserts. Devices, systems and methods for contraceptive approaches have been described in various patents and patent applications assigned to the present assignee. For example, U.S. Pat. No. 6,526,979, U.S. Pat. No. 6,634,361, and U.S. patent application Ser. No. 12/605,304 published as U.S. Publication No. 2011/0094519, describe transcervically introducing an insert (also referred to as implant and device) through an ostium of a fallopian tube and mechanically anchoring the insert within the fallopian tube. One example of such an assembly is known as “Essure”®, manufactured by Conceptus, Inc. of Mountain View, Calif. Tissue in-growth into the Essure® insert induces long-term contraception and/or permanent sterilization.

Referring to FIG. 1, an insert may access an ostium 106 of a fallopian tube through a cervix 102. A delivery system may be used to carry the insert through cervix 102 into a uterus 104 toward ostium 106. The insert may be a contraceptive implant held within a delivery catheter of the delivery system. For example, the delivery catheter may include an outer catheter sheathing the implant prior to deployment. From within uterus 104, the delivery system may be advanced until the insert is located beyond ostium 106 in an intramural portion 108 of the fallopian tube. Intramural portion 108 leads into an isthmus segment 110 of the fallopian tube. The intramural portion 108 and isthmus segment 110 generally have a combined length of about 3-4 cm. Furthermore, intramural portion 108 and isthmus segment 110 generally have an inner diameter of about 0.5-2 mm along their length. As a result, intramural portion 108 and isthmus segment 110 provide a suitable landing for delivery of the insert into the fallopian tube. Alternatively, the insert may be delivered into an ampulla segment 112, but given that the ampulla segment 112 inner diameter can vary to be up to about 1 cm at a distal end, securing an insert in that region is difficult. Furthermore, delivering an insert into the ampulla segment 112 may be accompanied by increased health risks such as an increase in the risk of an ectopic pregnancy.

An endoscope may be used to facilitate transcervical passage of the delivery system into a patient and to view placement of the delivery system through ostium 106. Once a physician has positioned the delivery system within the fallopian tube the insert may be deployed from the delivery catheter into the fallopian tube. The insert anchors within intramural portion 108 and/or isthmus segment 110 to occlude the fallopian tube and to prevent the passage of an ovum from ampulla 112 to uterus 104.

### SUMMARY OF THE DESCRIPTION

An occlusion device having a hollow body, a plurality of caps, and an expandable layer is disclosed. In an embodiment, the hollow body includes an outer surface and a passage with a proximal and distal end. The hollow body can be of any shape, e.g., ellipsoidal, cylindrical, spherical, etc. The plurality of caps may cover the passage ends to enclose the passage within the hollow body. The expandable layer may be coupled with the outer surface.

In an embodiment, one or more expandable anchors may also be coupled with the outer surface. For example, an anchor may include an expandable coil having an end coupled with the outer surface and another unattached end that expands radially apart from the coupled end when the expandable coil is unconstrained.

In an embodiment, the outer surface may be shaped in a convex, cylindrical, or concave manner. The expandable layer on the outer surface may include a non-slip surface to facilitate anchoring. For example, the expandable layer may include a hydrogel to promote tissue in-growth and adhesion. Alternatively, the expandable layer may include a shape memory polymer (SMP) or chemical foam structure with shape memory to facilitate anchoring and promote tissue in-growth and adhesion. Furthermore, the hydrogel or shape memory foam/polymer structure may be bioresorbable. In an embodiment, the occlusion device length may be between about 2 and 35 mm.

In an embodiment, the plurality of caps may be integrally formed with the hollow body or separately formed. Integrally formed caps may include a penetrable wall configured to be pierced by a guidewire. For example, the penetrable wall may be thinner than an adjacent wall of the hollow body or may include penetrable features, such as depressions or perforations. In an embodiment, the plurality of caps may be interconnected by a connector member.

An elongated flexible tail may be coupled with one of the plurality of caps. For example, the flexible tail may trail away from a proximal cap when the occlusion device is implanted in a fallopian tube. To aid visualization of the occlusion device, the enclosed passage may be filled with contrast agent.

A method of using the occlusion device includes delivering the occlusion device into a fallopian tube and expanding the expandable layer until it contacts and occludes the fallopian tube. The plurality of caps prevents migration of ova through the passage. The anchors may further secure the occlusion device to the fallopian tube. Thus, contraception may be achieved after deployment of the occlusion device. The method may further include opening the plurality of caps, for example, by dislodging or piercing one or more of the caps, to open the passage to the surrounding fallopian tube. Thus, ova are allowed to migrate through the passage to the uterus, and contraception can be reversed.

### BRIEF DESCRIPTION OF THE DRAWINGS

The present invention is illustrated by way of example and not limitation in the figures of the accompanying drawings in which like references indicate similar, but not necessarily identical, elements.

FIG. 1 is a pictorial illustration of a human female reproductive system;

FIG. 2 is a side view illustration of an occlusion device having an ellipsoid hollow body in accordance with an embodiment of the invention;

FIG. 3 is a cross-sectional view, taken about line A-A of FIG. 2, illustrating an occlusion device in accordance with an embodiment of the invention;

FIG. 4 is a side view illustration of an occlusion device having an expanded expandable layer in accordance with an embodiment of the invention;

FIG. 5 is a side view illustration of an occlusion device having a cylindrical hollow body in accordance with an embodiment of the invention;

FIG. 6 is a cross-sectional view, taken about line B-B of FIG. 5, illustrating an occlusion device in accordance with an embodiment of the invention;

FIG. 7 is a side view illustration of an occlusion device having an anchor feature in accordance with an embodiment of the invention;

FIG. 8A is a side view illustration of an occlusion device having an expanded expandable layer and expanded coil anchor features in accordance with an embodiment of the invention;

FIG. 8B is a side view illustration of an occlusion device having an expanded expandable layer and expanded spider member and stent-like anchor features in accordance with an embodiment of the invention;

FIG. 9 is a side view illustration of an occlusion device having an opened proximal cap in accordance with an embodiment of the invention;

FIG. 10 is a side view illustration of an occlusion device having an opened distal cap in accordance with an embodiment of the invention;

FIG. 11 is a side view illustration of an occlusion device having an opened passage in accordance with an embodiment of the invention;

FIGS. 12A-12E are various views of cap portions of an occlusion device in accordance with an embodiment of the invention;

FIGS. 13A-13C are various views of tail portions of an occlusion device in accordance with an embodiment of the invention;

FIG. 14 is a pictorial illustration of an endoscope accessing an ostium of a fallopian tube in accordance with an embodiment of the invention;

FIGS. 15A-15D are pictorial illustrations of delivery of an occlusion device to occlude a fallopian tube and to effect contraception in accordance with an embodiment of the invention; and

FIGS. 16A-16C are pictorial illustrations of unblocking of the fallopian tube to reverse contraception in accordance with an embodiment of the invention.

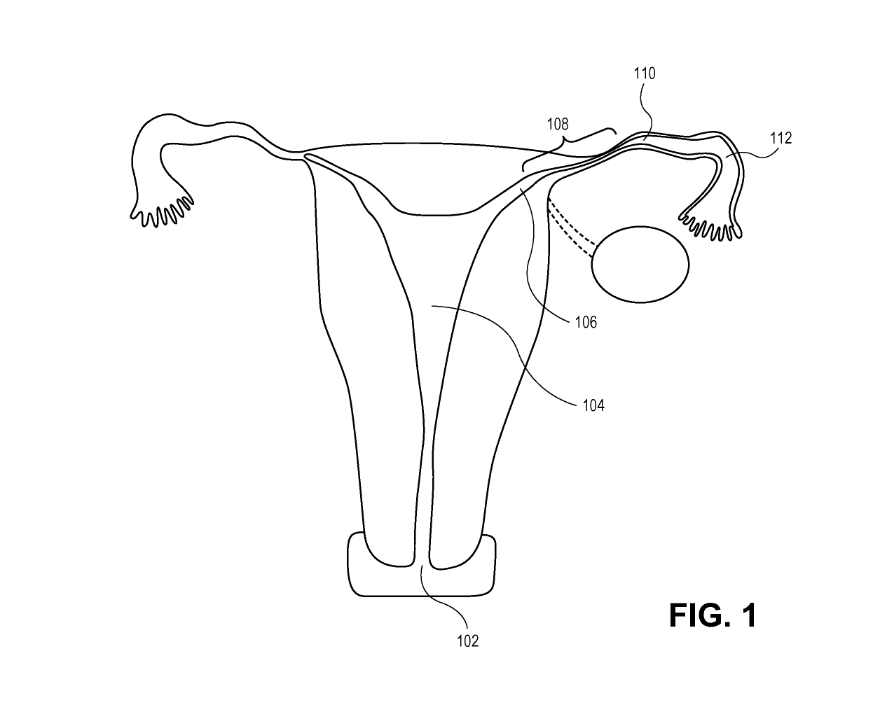

### DETAILED DESCRIPTION

Various embodiments and aspects of the invention will be described with reference to details discussed below, and the accompanying drawings will illustrate the various embodiments. The following description and drawings are illustrative of the invention and are not to be construed as limiting the invention. Numerous specific details are described to provide a thorough understanding of various embodiments of the invention. However, in certain instances, well-known or conventional details are not described in order to provide a concise discussion of embodiments of the present invention.

Reference in the specification to “one embodiment” or “an embodiment” means that a particular feature, structure, or characteristic described in conjunction with the embodiment can be included in at least one embodiment of the invention. The appearances of the phrase “in one embodiment” in various places in the specification do not necessarily all refer to the same embodiment. Although the processes are described below in terms of some sequential operations, it should be appreciated that some of the operations described may be performed in a different order. Moreover, some operations may be performed simultaneously rather than sequentially.

It is to be understood that embodiments of the current invention may be used in gynecological, as well as non-gynecological, target anatomies. For example, many target anatomies may benefit from procedures using the current invention, including gynecological anatomies such as the fallopian tubes, and non-gynecological target anatomies, such as the vas deferens, e.g., during a male sterilization procedure, or arteries, e.g., during a vascular intervention. Thus, while the current invention is applicable to surgeries that employ endoscopes, such as angioscopy, arthroscopy, bronchoscopy, and hysteroscopy, to name a few, it is equally applicable to surgeries that employ other delivery systems, such as vascular intervention procedures.

In an aspect, an occlusion device facilitates contraception by occluding a target anatomy, such as a fallopian tube, to prevent the passage of reproductive cells, such as ova, across the occlusion device. In an embodiment, an expandable layer fills a space between a fallopian tube wall and a hollow body to prevent passage of an ovum around the hollow body. Furthermore, the hollow body may include one or more caps that enclose a passage within the hollow body and prevent the ovum from traveling through the hollow body. Thus, migration of the ovum from the ampulla to the uterus is impeded to effect contraception.

In another aspect, the occlusion device facilitates reversible contraception by reopening the occluded target anatomy to allow the passage of reproductive cells. In an embodiment, the one or more caps of the hollow body may be removed, pierced, or otherwise opened to uncover and disclose the passage. Thus, the fallopian tube may become unblocked and migration of the ovum from the ampulla through the hollow body into the uterus is enabled to reverse contraception.

In another aspect, the occlusion device that facilitates reversible contraception anchors within the target anatomy. In an embodiment, the occlusion device may include one or more anchors that expand within the fallopian tube to secure the hollow body to the fallopian tube. Anchoring may be achieved through non-slip surface features of the expandable layer. Alternatively, anchoring may be achieved through expanding structures and/or coatings that provide acute and long-term adherence to the fallopian tube. Thus, the anchored occlusion device may remain intact within the fallopian tube to ensure contraception is maintained until reversal of contraception is intended.

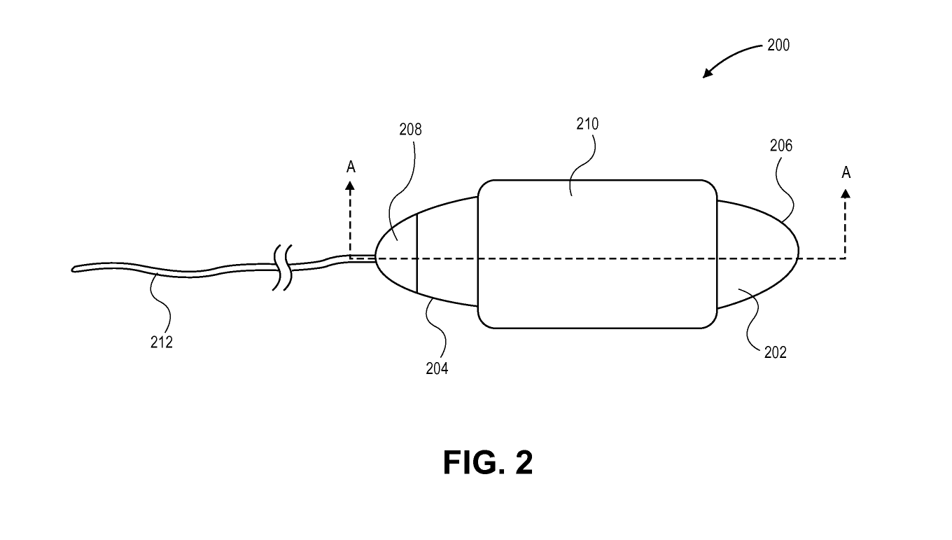

Referring to FIG. 2, a side view of an occlusion device having an ellipsoid hollow body is shown in accordance with an embodiment of the invention. In an embodiment, occlusion device 200 includes a centrally located hollow body 202 having a shell configuration. For example, hollow body 202 may be formed as a shell capsule with a thin wall, such that a cavity or passage is located within hollow body 202.

Hollow body 202 may be configured to facilitate placement within a fallopian tube. For example, hollow body 202 may be formed from a material that is able to conform to arcuate fallopian tube anatomies and to accommodate motion of the fallopian tubes that occur during normal patient movement. Thus, in an embodiment, hollow body 202 may be formed from a flexible and resilient material, such as a copolymer of ethylene and polypropylene. Alternatively, portions of hollow body 202 may incorporate polyurethane, silicone, butyl rubber, or other elastomeric materials.

In addition to being flexible, hollow body 202 may have sufficient radial strength to resist radial collapse under loads applied by the fallopian tube. More specifically, normal patient movement may result in bending and compressive loads applied to hollow body 202, but hollow body 202 may be formed with sufficient radial strength and/or resilience to either not collapse or to not permanently deform under such loads.

Although hollow body 202 may be formed from biostable materials, such as those listed above, in an alternative embodiment, hollow body 202 may be bioabsorbable or bioresorbable. For example, in an embodiment, hollow body 202 may be formulated to degrade within a fallopian tube over a set period of time such that hollow body 202 initially occludes the fallopian tube but is eventually resorbed by the fallopian tube. Following resorption, hollow body 202 may no longer impede migration of ova. As an example, hollow body 202 may be formed from a biodegradable plastic, such as a polymer containing polyglycolic acid or polylactic acid configured to degrade either by bulk erosion or surface erosion. Hollow body 202 may be tuned to reopen through this erosion within a predetermined time frame, e.g., approximately two to three years after implantation. Thus, occlusion device 200 will effect contraception over a period of about three years, but following that time, degradation of occlusion device 200 will reverse contraception.

Hollow body 202 may also be sized to facilitate placement within a fallopian tube. For example, hollow body 202 may have a diameter that conforms closely to the average diameter of intramural portion 108 or isthmus segment 110. Accordingly, hollow body 202 may have a maximum diameter between about 0.5 to 2 mm. Furthermore, the length of hollow body 202 may be chosen to prevent hollow body 202 from upending within the fallopian tube. Hollow body 202 length may be chosen to be at least as long as is needed to mitigate the risk that collateral channels will carry an ovum around implanted occlusion device 200 and thereby negate the occlusion device-induced contraception. Accordingly, in an embodiment, hollow body 202 may have a length between about 2 to 35 mm. More specifically, hollow body 202 may have a length from a proximal end to a distal end of about 10 mm.

In an embodiment, an expandable layer 210 may be located on an outer surface 204 of hollow body 202. Expandable layer 210 may be formed from a material that swells upon introduction into a physiological environment, such as the fallopian tube. For example, in an embodiment, expandable layer 210 may be formed from a hydrogel material. Hydrogels can be formed from covalently or non-covalently cross-linked materials, and can be non-degradable (“biostable”) in a physiological environment or broken down by natural processes within the body (“biodegradable”, “bioabsorbable”, or “bioresorbable”). Accordingly, in several embodiments, expandable layer 210 may be a polyacrylamide, polyethylene glycol (PEG), or polyvinylpyrrolidone (PVP) based hydrogel. Thus, a hydrogel expandable layer 210 may have a substantially unswollen state as well as a swollen state. Expandable layer 210 may be maintained in the substantially unswollen state during delivery to the fallopian tube, but after delivery, expandable layer 210 may swell against the fallopian tube wall. Alternatively, expandable layer 210 may be formed from a shape memory polymer (SMP) material such as polyurethane-based shape memory polymers or polymer foams that have excellent biocompatibility and tunable glass transition temperatures for shape restoration/self-deployment when inserted in the human body.

A hydrogel used in expandable layer 210 may, in one embodiment, be made to be radiopaque by incorporating a radiopaque filler, such as iodine or heavy metals and heavy metal compounds, e.g., barium sulfate, platinum, tungsten, gold, or iridium-based contrast material, into the hydrogel. Increasing hydrogel radiopacity promotes visibility of the implanted occlusion device 200 under fluoroscopy. Similarly, visibility of the implanted occlusion device 200 may be modified to accommodate other imaging modalities. For example, visibility of the hydrogel and occlusion device 200 under ultrasound imaging may be improved by incorporating microbubbles within the hydrogel.

Expandable layer 210 may be coupled with outer surface 204 to stabilize hollow body 202 within the fallopian tube when expandable layer 210 expands therein. In an embodiment, expandable layer 210 may be spray coated or dip coated over outer surface 204. Alternatively, expandable layer 210 may be separately formed and then coupled with hollow body 202 using an adhesive layer between outer surface 204 and expandable layer 210. For example, expandable layer 210 may be cross-linked separately or injection molded separately before being coupled with hollow body 202. Thus, after expandable layer 210 is coupled with outer surface 204, it may be securely attached to hollow body 202.

Occlusion device 200 may include one or more caps, such as distal cap 206 and proximal cap 208, which are located at either end of hollow body 202. The caps may be integrally formed with hollow body 202, or formed separately and then coupled with hollow body 202. In FIG. 2, for example, distal cap 206 is integrally formed with hollow body 202 and provides a distal end of hollow body 202. By contrast, proximal cap 208 is formed separately from hollow body 202 and is coupled with hollow body 202. Although distal cap 206 may be a portion of hollow body 202 while proximal cap 208 may be separate from hollow body 202, in either case, distal cap 206 and proximal cap 208 cover an end of hollow body 202 and therefore enclose the cavity that is located within hollow body 202. Thus, while distal cap 206 and proximal cap 208 are coupled with hollow body 202 and enclose the cavity, an ovum is prevented from passing through hollow body 202 from one end to another end.

In an embodiment, occlusion device 200 may include a tail 212 trailing from proximal cap 208 or hollow body 202. For example, tail 212 may be a flexible suture having a distal end attached to proximal cap 208 and a proximal end trailing freely away from the hollow body 202. Tail 212 may be sized and configured to allow for it to trail into uterus 104 while hollow body 202 is located within a fallopian tube. For example, tail 212 may have a diameter less than the average diameter of intramural portion 108 and a length greater than an average length of intramural portion 108. In an embodiment, tail 212 may have a diameter of less than about 1 mm and a length of at least about 1 cm. Tail 212 may include a flexible suture, which may be a braided or monofilament type suture, for example. Thus, the size and structure of tail 212 provides for a flexible, and in some cases limp, elongated marker that conforms to the fallopian tube and hangs from ostium 106 into uterus 104. Accordingly, tail 212 can be visually identified by an endoscope inserted within uterus 104.

Several features may be incorporated to promote the visibility of tail 212 under both optical and x-ray imaging modalities. For example, tail 212 may be colored to visually contrast with tissue, e.g., by a surface treatment that is itself visible when illuminated and viewed employing a hysteroscope. For example, tail 212 may be coated with a dye-loaded hydrophilic coating that liquefies when it contacts the tissue surface and stains the tissue. Preferred non-toxic dyes for staining the tissue of the ostium 106 or for coloring sutures include methylene blue dye (a benign chemical often used in conjunction with hysteroscopic fertility tests), FD&C BLUE dyes 3 and 6, eosin, and indocyanine green. Green or blue dyes substantially contrast with the uterine wall tissue color. In another embodiment, tail 212 may include a radiopaque ink or dye either painted on an outer surface 204 or incorporated within tail 212. Suitable radiopaque inks include inks containing barium sulfate, gold, or other dense materials that are visible under fluoroscopy.

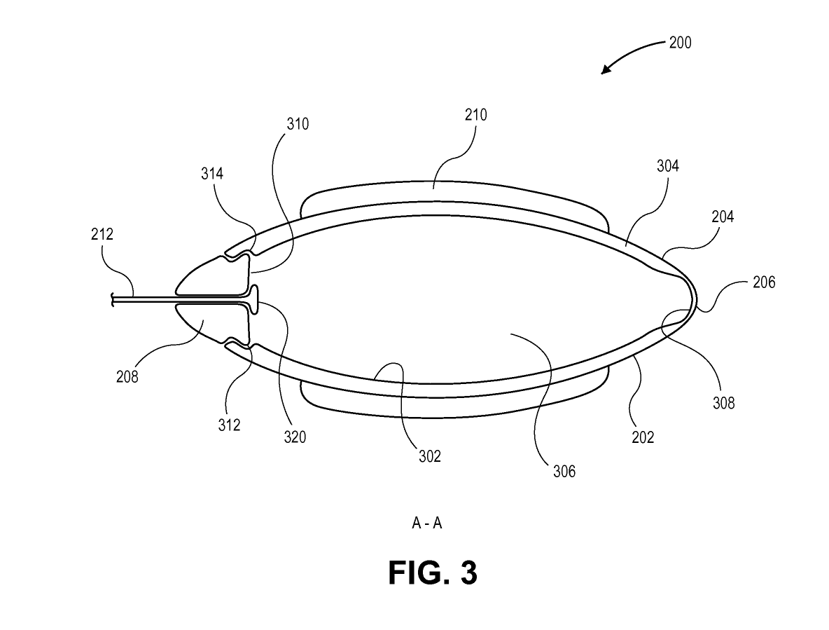

Referring to FIG. 3, a cross-sectional view, taken about line A-A of FIG. 2, illustrates an occlusion device in accordance with an embodiment of the invention. A distal end 320 of tail 212 may be coupled with proximal cap 208 by placing tail 212 through a bore formed through proximal cap 208. In an embodiment, tail 212 may be secured in the bore using an adhesive. In an alternative embodiment, a distal end 320 of tail 212 may be stamped to cause a localized increase in the tail 212 outer diameter and thereby prevent tail 212 from being pulled out through the bore in proximal cap 208.

Passage 306 may be defined by the cavity within hollow body 202. More specifically, an inner surface 302 of hollow body 202 may be a closed surface that defines a space through which passage 306 is located. Thus, in an embodiment, passage 306 may have a volume that substantially conforms with the volume of hollow body 202. For example, hollow body 202 may be a thin-walled shell with a body wall 304 between outer surface 204 and inner surface 302. In an embodiment, body wall 304 may have a uniform thickness of about 0.1 to 0.25 mm. Accordingly, the cavity within hollow body 202 may be substantially similar to a volume within inner surface 302, and passage 306 may be considered to encompass the entire cavity. In other embodiments, body wall 304 may not have a uniform thickness or may include additional features, such as ribbing, and therefore the volume of the cavity may be reduced. However, even in these alternative embodiments, passage 306 may be defined as passing along a path from a distal end 308 to a proximal end 310. In an embodiment, passage 306 has an opening at distal end 308 and an opening at proximal end 310. As shown in FIG. 3, distal cap 206 and proximal cap 208 may cover those openings, thereby enclosing passage 306 relative to the surrounding environment.

In an embodiment, distal cap 206 may be integrally formed with hollow body 202, and more specifically, may include material that is contiguous with body wall 304. However, distal cap 206 may be formed such that it is more susceptible to puncture as compared to body wall 304. For example, distal cap 206 may be thinner than adjacent body wall 304. As an example, whereas body wall 304 may have a uniform thickness of about 0.1 to 0.25 mm, distal cap 206 may have a thickness of about 0.05 mm to 0.15 mm. Thus, distal cap 206 covers an opening of passage 306 at distal end 308, but also provides a structure that can be preferentially punctured to expose passage 306 to the surrounding environment.

In an embodiment, proximal cap 208 may be separately formed from hollow body 202, and more specifically, may be separately formed and then coupled with body wall 304. However, proximal cap 208 may be formed such that it is removable from hollow body 202. For example, proximal cap 208 may include a bulge 312 that fits within a detent 314 formed in body wall 304. Bulge 312 and detent 314 may be sized to mate with each other such that proximal cap 208 can be snapped into, and retained against, hollow body 202 until a sufficient load is applied to disengage bulge 312 from detent 314. For example, tail 212 may be pulled to disengage bulge 312 from detent 314 and remove proximal cap 208 from hollow body 202. Thus, proximal cap 208 covers an opening of passage 306 at proximal end 310, but also provides a structure that can be manipulated to expose passage 306 to the surrounding environment.

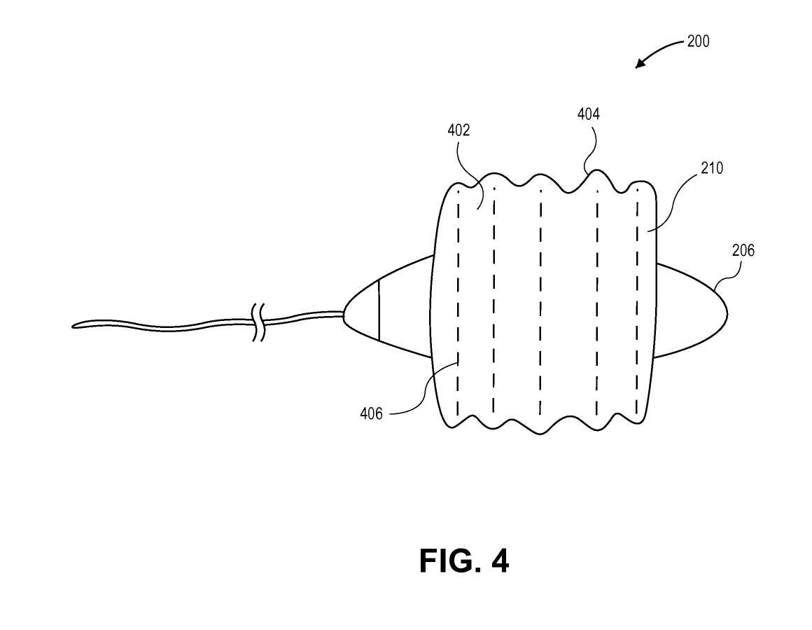

Referring to FIG. 4, a side view of an occlusion device having an expanded expandable layer is shown in accordance with an embodiment of the invention. As described above, expandable layer 210 may include a hydrogel that swells upon delivery into a physiological environment. Thus, upon delivery into a fallopian tube, expandable layer 210 can increase in volume and fill a space between hollow body 202 and the fallopian tube. However, in addition to swelling and occluding fallopian tube, expandable layer 210 may also include a non-slip surface 402 that encourages anchoring of the occlusion device 200 within the fallopian tube. For example, a hydrogel material in expandable layer 210 may facilitate tissue in-growth and epithelialization. Such in-growth results in adhesion between expandable layer 210 and the fallopian tube.

In an embodiment, non-slip surface 402 of expandable layer 210 may also include one or more structural features that encourage anchoring. For example, expandable layer 210 may include one or more corrugation 404 defined by an area of increased diameter adjacent to areas of decreased diameter, e.g., an undulating, wavy, or jagged surface. Corrugation 404 facilitates gripping of the fallopian tube wall. Given that the fallopian tube wall is generally formed of a multi-layered resilient muscular structure, the fallopian tube will conform to the peaks and valleys of the non-slip surface 402 and thereby resist axial motion of the expandable layer 210. In addition to a corrugated surface, non-slip surface 402 may have one or more other anchor features, such as barbs, protrusions, ribs, etc., that facilitate the same gripping effect described above.

In an embodiment, non-slip surface 402 may include one or more surface treatments to encourage anchoring. For example, in addition to tissue in-growth within a hydrogel, further surface treatment may be used to promote tissue adhesions. For example, expandable layer 210 may be coated with a biocompatible adhesive to facilitate adhesion between the hydrogel of expandable layer 210 and tissue of the fallopian tube. A wide range of bioadhesives may be used for this purpose, but as an example, bioadhesives such as chitosan, which have found applications for wound treatment, may be used.

In an embodiment, non-slip surface 402 may include structural features separate from the native expandable layer 210 material or surface treatment thereof. For example, in addition to a corrugated or surface treated hydrogel surface, additional components may be coupled with expandable layer 210 to promote tissue ingrowth and adhesion to the fallopian tube. In an embodiment, a meshlike material such as polyester fibers or other fibers or furs which are designed to promote tissue ingrowth may be incorporated into or around expandable layer 210. Such meshlike materials may promote tissue ingrowth and corresponding adhesion between occlusion device 200 and the fallopian tube. In another embodiment, a thin wire may be wrapped around expandable layer 210 in a coil fashion to facilitate epithelialization surrounding expandable layer 210, and to secure occlusion device 200 within the fallopian tube.

In an embodiment, a frame 406 may be incorporated within expandable layer 210 to provide structural support. For example, frame 406 may be a coil or mesh member, such as a thin metallic wire, inserted or deposited within expandable layer 210. Frame 406 may be able to expand as expandable layer 210 swells or self-deploys (shape memory behavior), but will also provide a scaffold for expandable layer 210 to bind with. Thus, swellable materials like hydrogel or self-expanding memory material/structure incorporated in expandable layer 210 are scaffolded to better resist decomposition over time.

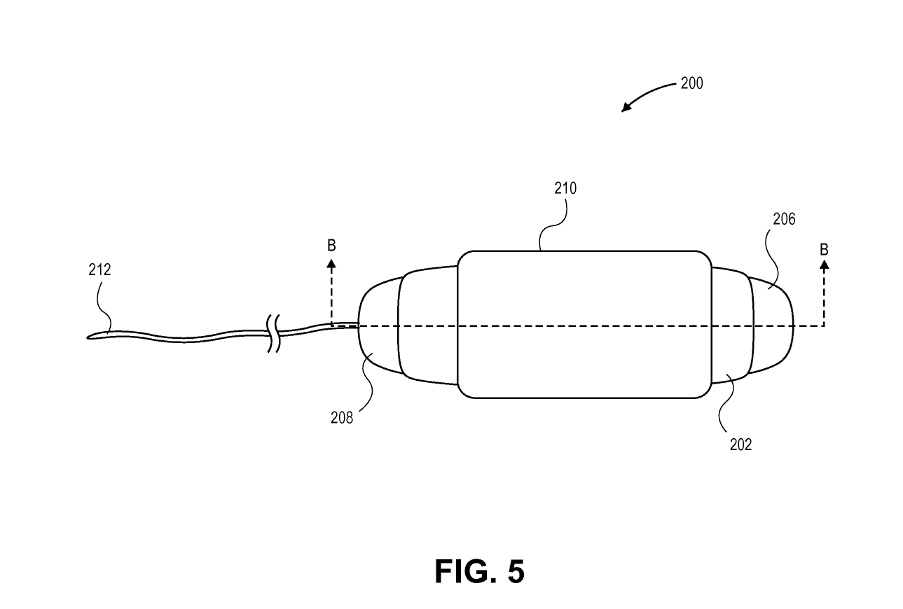

Referring to FIG. 5, a side view of an occlusion device having a cylindrical hollow body is shown in accordance with an embodiment of the invention. Thus, hollow body 202 is not limited to an ellipsoid configuration. On the contrary, hollow body 202 may be shaped with a convex shape, such as an ellipsoid, or it may have numerous other shapes, such as cylindrical or concave. A concave shape may include diameters at the ends of hollow body 202 that are greater than a medial diameter. In one embodiment, hollow body 202 may be spherical, with an outer diameter of about 0.5 to 2 mm. In another embodiment, hollow body 202 may be cylindrical with diameter and length dimensions similar to those described for the ellipsoid configuration above. Regardless of the shape of hollow body 202, occlusion device 200 may include similar components, i.e., hollow body 202, expandable layer 210, distal cap 206, proximal cap 208, and tail 212.

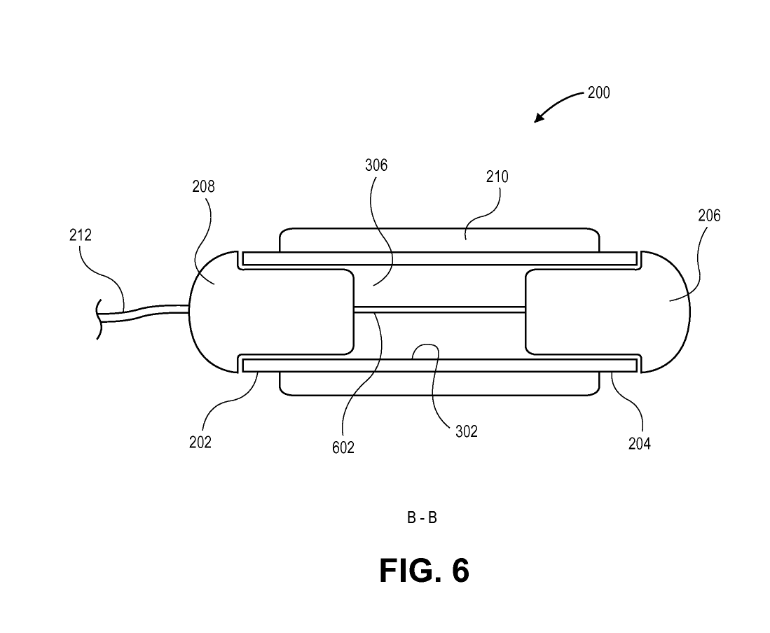

Referring to FIG. 6, a cross-sectional view, taken about line B-B of FIG. 5, illustrates an occlusion device in accordance with an embodiment of the invention. Regardless of the shape of hollow body 202, occlusion device 200 may include a channel such as passage 306. However, the shape of hollow body 202 defines the cavity volume through which passage 306 extends, and thus, a cylindrical hollow body 202 with a uniform diameter over its length will have a larger passage 306 volume than an ellipsoid hollow member of equivalent diameter and length.

Distal cap 206 and proximal cap 208 may engage the cavity within hollow body 202 to plug passage 306. In an embodiment, both distal cap 206 and proximal cap 208 are formed separately from hollow body 202 and cover openings in hollow body 202 to enclose passage 306. Distal cap 206 and proximal cap 208 may be linked by a connector 602. For example, connector 602 may be a braided or monofilament cord attached at either end to a different cap. Alternatively, connector 602 may be contiguously formed with tail 212, and thus may be an extension of tail 212 that runs through proximal cap 208 to distal cap 206. In either case, as proximal cap 208 is pulled away from hollow body 202, a tensile load will be applied to connector 602, which then pulls distal cap 206.

In an embodiment, as proximal cap 208 and distal cap 206 are pulled by tail 212 and/or connector 602, a tensile load may be distributed through their masses to cause them to stretch and unseal from passage 306. Stretching of the caps may be facilitated by forming the caps from an elastomeric material, such as a silicone. Thus, as tail 212 is pulled, proximal cap 208 may disengage and be removed from passage 306 and distal cap 206 may be pulled through passage 306 until it is finally removed from hollow body 202. Accordingly, a single pulling force applied to tail 212 may result in dislodgement of both proximal cap 208 and distal cap 206 and exposure of passage 306 to the surrounding environment.

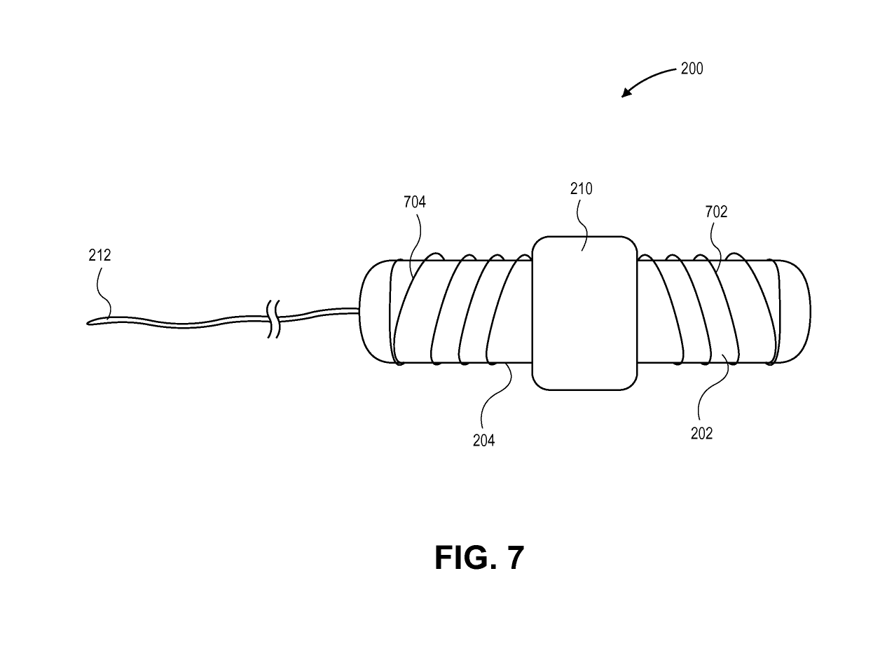

Referring to FIG. 7, a side view of an occlusion device having an anchor feature is shown in accordance with an embodiment of the invention. In an embodiment, occlusion device 200 includes one or more anchors to further facilitate securement of occlusion device 200 to a fallopian tube. Occlusion device 200 may include a distal anchor 702 and/or a proximal anchor 704 on either side of expandable layer 210. In an embodiment, anchors 702, 704 may actually be proximal and distal portions of the same structure. For example, a single coil may wrap around hollow body 202 and be sandwiched against outer surface 204 by expandable layer 210.

In a first configuration to facilitate delivery of occlusion device 200, the anchors may be in a low profile configuration, i.e., a small diameter. For example, proximal anchor 704 and distal anchor 702 may be formed from coil structures that are wound down to conform closely to outer surface 204 of hollow body 202 in the delivery configuration. Anchors 702, 704 may be retained in the low profile configuration either through internal material structure, e.g., in a martensitic phase, or by an external constraint, e.g., a sheath or catheter placed around the anchors.

In an embodiment, additional features may be incorporated with the anchor structures to encourage anchoring. For example, a meshlike material such as polyester fibers or other fibers or furs which are designed to promote tissue ingrowth may be incorporated into or around proximal anchor 704 and/or distal anchor 702 to promote tissue ingrowth and adhesion between occlusion device 200 and the fallopian tube. Additionally or alternatively, adhesion-promoting coatings may be incorporated into or over proximal anchor 704 and/or distal anchor 702 to promote tissue ingrowth and adhesion. For example, proximal anchor 704 and/or distal anchor 702 may be coated with a biocompatible adhesive to facilitate adhesion.

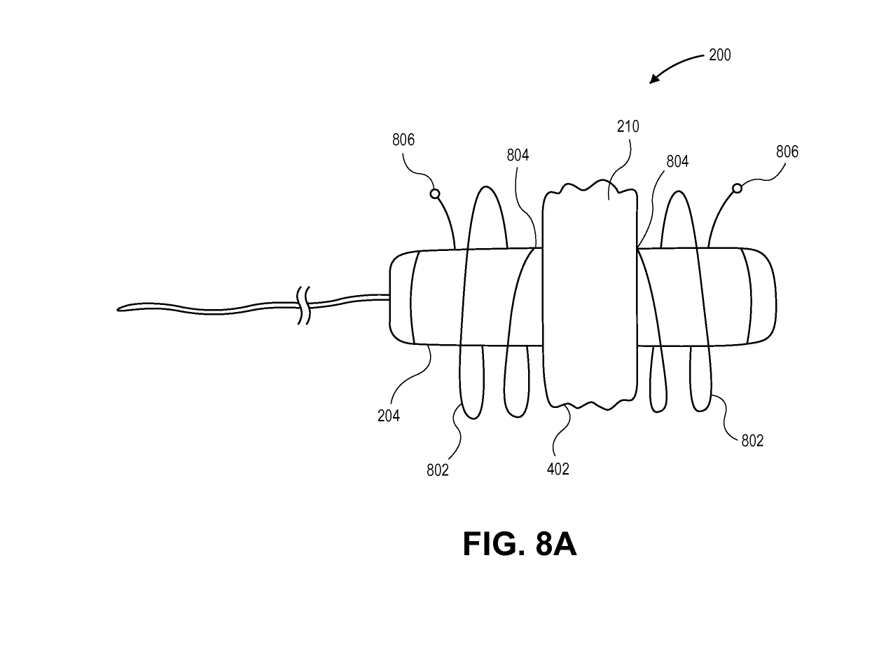

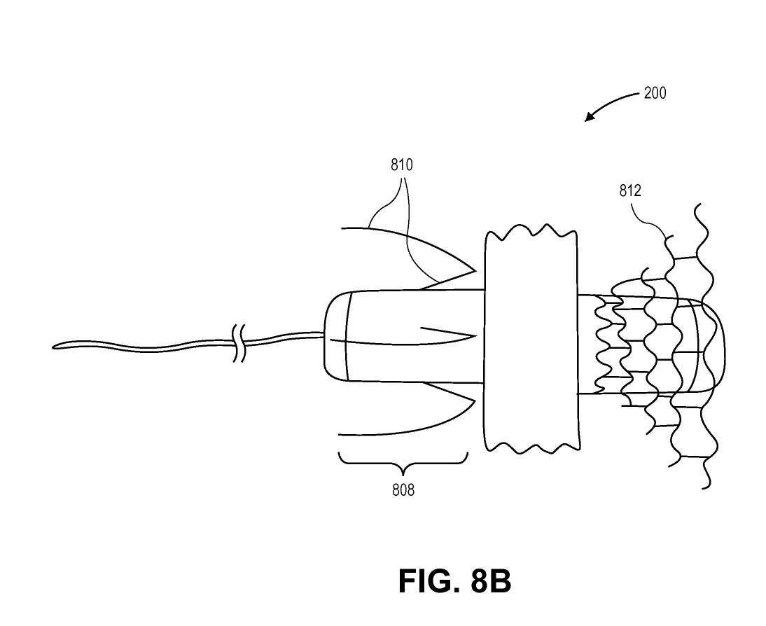

Referring to FIG. 8A, a side view of an occlusion device 200 having an expanded expandable layer and expanded coil anchor features is shown in accordance with an embodiment of the invention. Distal anchor 702 and proximal anchor 704 may include an expandable coil 802 constructed of a thin wire that unwinds upon deployment to increase in diameter and contact fallopian tube. Accordingly, expandable coil 802 may have an attached end 804 that is coupled with outer surface 204 and an unattached end 806 that extends freely away from attached end 804. As expandable coil 802 unwinds, unattached end 806 may become radially offset from attached end 804, relative to a central axis passing through hollow body 202. In addition, unattached end 806 and at least a portion of expandable coil 802 may increase to a diameter greater than a swollen diameter of expandable layer 210, to create a mechanical gripping force against a fallopian tube, which contributes to the adhesion between expandable layer 210 and the fallopian tube.

Expandable coil 802 may be formed from conventional metals and polymers, such as stainless steel, cobalt-chrome, polyethylene, etc., and sized and configured to prevent material yield when wound against hollow body 202. As a result, upon release of expandable coil 802, the coil unwinds and springs outward. Alternatively, expandable coil 802 may be formed from a shape memory material, such as nickel-titanium, and configured to expand under physiological conditions to a larger, unwound configuration due to a material phase transformation. In an embodiment, expandable coil 802 formed from nickel-titanium may be encapsulated or sheathed within a biocompatible, biostable polymer, e.g., by co-extrusion, dip coating, or placing a tubular sheath over expandable coil 802. Some data suggests an incidence of nickel hypersensitivity in patients, and thus, encapsulation may avoid expandable coil 802 contacting and adversely affecting patient tissue.

Referring to FIG. 8B, a side view of an occlusion device having an expanded expandable layer and expanded spider member and stent-like anchor features is shown in accordance with an embodiment of the invention. Stent-like anchors 812 or spider members 808 provide alternative anchor structures. Examples of spider members 808 which may be used are shown in co-assigned U.S. Pat. No. 8,235,047. Each spider member 808 may include at least two spider arms 810. Spider arms 810 are generally made up of two members that are folded against each other in a non-expanded state. Fibers may also be interwoven between and diametrically across spider arms 810 to promote tissue ingrowth as described above. Stent-like anchors 812 may be used that have a scaffold structure that is expandable from a low profile to an expanded profile. Spider members 808 and stent-like anchors 812 may be formed from the same materials and configured to expand in a fashion similar to that described for expandable coil 802 above. Although in FIG. 8B occlusion device 200 is shown including one spider member 808 and one stent-like anchor 812, occlusion device 200 may have any combination of distal and proximal anchors, including only one distal anchor or proximal anchor, or a distal anchor and proximal anchor of the same type, e.g., two spider members 808.

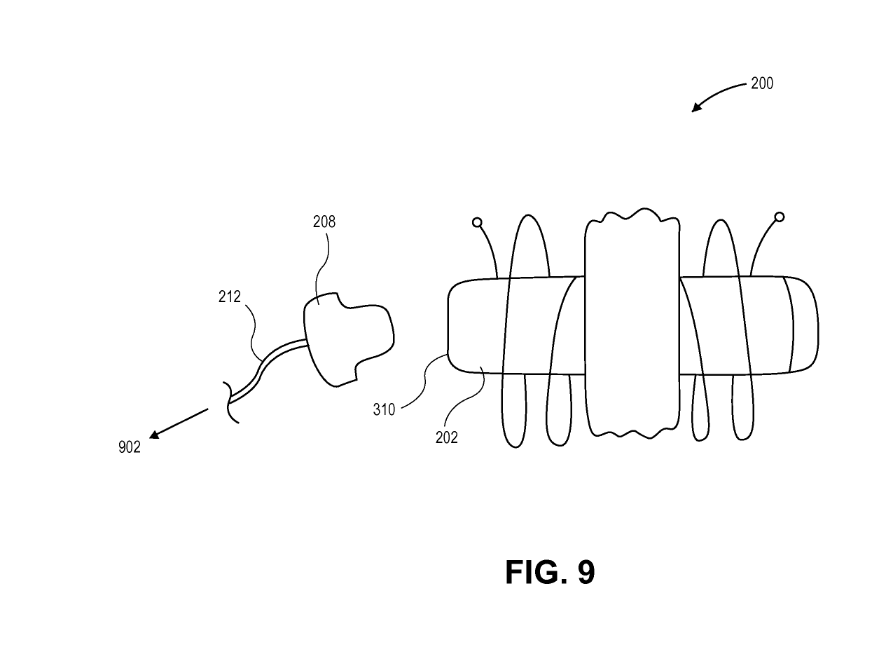

Referring to FIG. 9, a side view of an occlusion device having an opened proximal cap is shown in accordance with an embodiment of the invention. Proximal cap 208 may be removed from hollow body 202 to expose passage 306 to the surrounding environment. For example, a pulling force 902 may be applied to tail 212 to exert a removal load on proximal cap 208. As proximal cap 208 is removed, proximal end 310 of passage 306 will be opened. Thus, when occlusion device 200 is deployed within a fallopian tube, removal of proximal cap 208 will open passage 306 to a proximal portion of the fallopian tube.

Referring to FIG. 10, a side view of an occlusion device having an opened distal cap is shown in accordance with an embodiment of the invention. Distal cap 206 may be opened, for example, by pushing a cannulation member 1002 through distal cap 206 until distal cap 206 tears or breaks open. For example, cannulation member 1002 may be a guidewire, cannula, or a catheter device that is inserted through proximal end 310 of passage 306 until it contacts distal cap 206 at distal end 308 of passage 306. As cannulation member 1002 is pushed further, distal cap 206 eventually yields to open passage 306 to the surrounding environment. For example, when occlusion device 200 is deployed within a fallopian tube, pushing on distal cap 206 will open passage 306 to a distal portion of fallopian tube.

Opening distal cap 206 may also occur in a multi-stage process. For example, a guidewire may first be advanced through passage 306 to pierce distal cap 206 and then a catheter device may be delivered over the guidewire through distal cap 206 to widen the pierced opening. Guidewires are generally maneuverable, have small diameters, and can be formed with good column strength, which makes them well suited for accessing and passing through passage 306, and piercing distal cap 206. Furthermore, catheters are generally flexible, include blunted or atraumatic tips, and can be formed with an outer diameter that conforms closely to an inner diameter of hollow body 202, which makes them well suited for delivering over a guidewire to pass through hollow body 202 and to enlarge the openings at either end of the occlusion device 200. In an embodiment, an additional stage may include accessing opened passage 306 with a balloon catheter or stent delivery catheter and inflating a balloon within passage 306 to expand and/or scaffold hollow body 202, and to widen passage 306 even further. Thus, a multi-stage process for opening distal cap 206 may be performed with readily available medical equipment.

In an alternative embodiment, cannulation member 1002, or devices used to follow cannulation member 1002 in a multi-stage process, may include an atherectomy catheter or excimer laser ablation catheter. Such devices include a distal working end used to open blockages in body vessels and may be advanced through passage 306 and used to open distal cap 206. Furthermore, a guidewire or catheter device having an ultrasonic probe located in a distal tip may be used to apply ultrasonic energy and rapid vibration to pierce, puncture, crack, or otherwise open distal cap 206. For example, an ultrasonic probe may be particularly useful in opening a hard or rigid distal cap 206, e.g., where distal cap 206 is formed from a ceramic.

Referring to FIG. 11, a side view of an occlusion device having an opened passage is shown in accordance with an embodiment of the invention. In an embodiment, after deploying occlusion member in fallopian tube and securing occlusion member to fallopian tube with proximal anchor 704, distal anchor 702, and/or expandable layer 210, passage 306 is exposed to the surrounding environment by opening proximal cap 208 and distal cap 206. Thus, a passage axis 1102 is created through proximal end 310 and distal end 308 of passage 306, providing a pathway for an ovum to pass through hollow body 202 from a distal fallopian tube anatomy to a proximal fallopian tube anatomy. Passage axis 1102 need not be linear. For example, in an embodiment, passage 306 may follow a curved path corresponding to a curved fallopian tube or occlusion device 200, and in such case, passage axis 1102 may be curved as well.

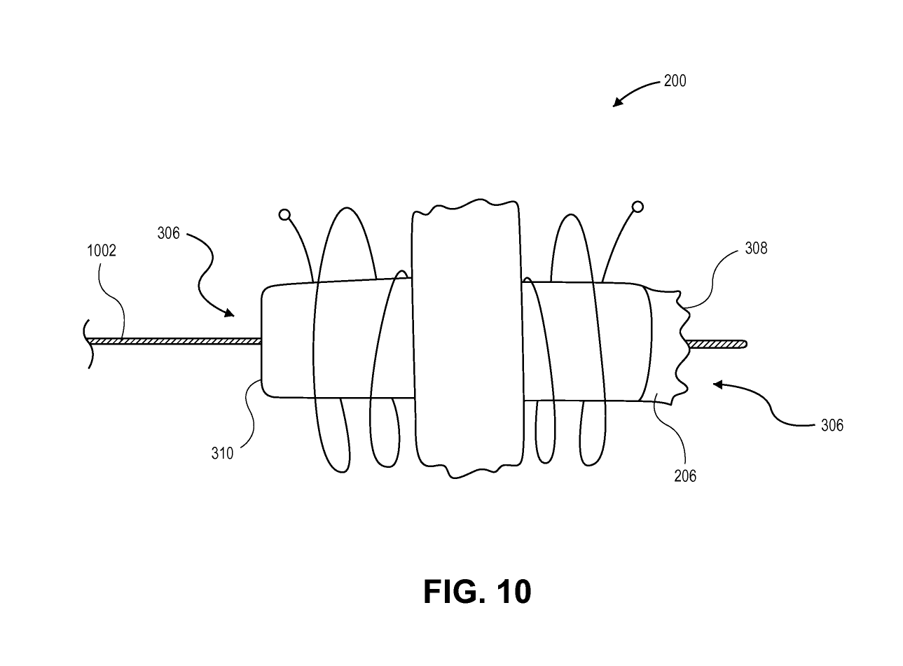

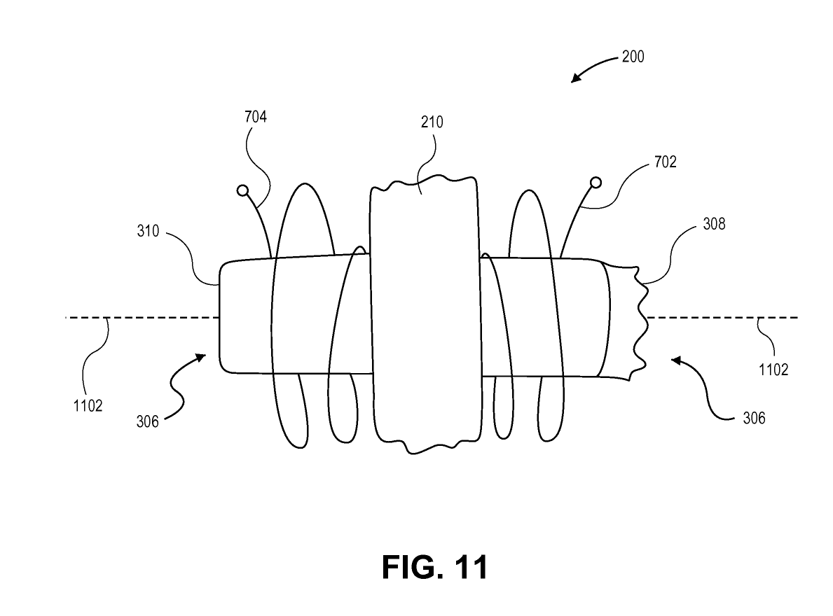

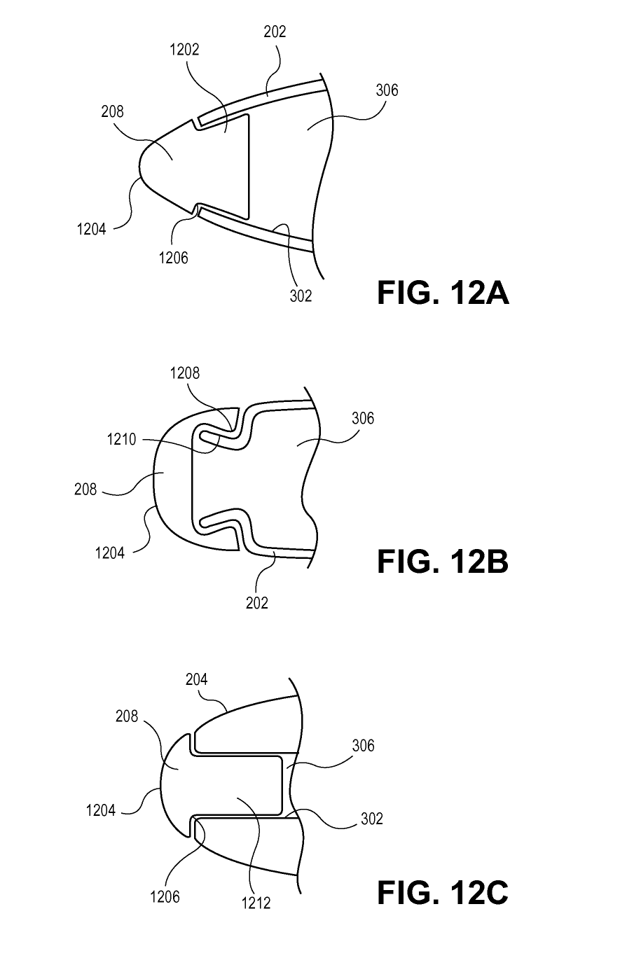

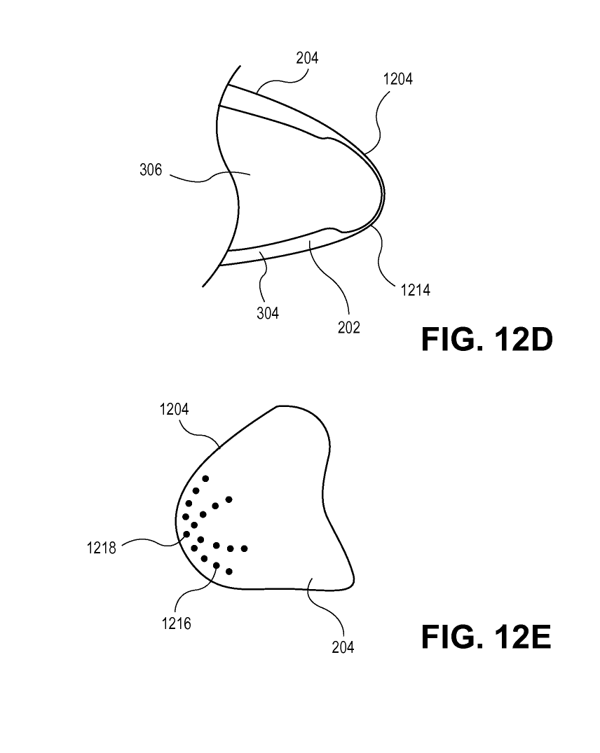

FIGS. 12A-12E provide various views of cap portions of an occlusion device in accordance with an embodiment of the invention. Although the various views are oriented to correspond to either proximal cap 208 or distal cap 206, the cap configurations may be used for either. Additionally, the various configurations are intended to be illustrative, and not exhaustive, of the numerous cap designs that may be used.

Referring to FIG. 12A, a proximal cap 208, distal cap 206 configuration having a flared retainer is shown in accordance with an embodiment of the invention. Hollow body 202 may include an inner surface 302 that tapers downward. For example, a thin-walled ellipsoid shell may have a tapering inner surface 302 near either end. In an embodiment, proximal cap 208 includes a flared portion that fits within passage 306 and flares outward to resist pulling the cap away from hollow body 202. Flare 1202 may be a revolved body, symmetric about a central axis, or it may be a non-symmetric body with one or more flaring elements. For example, flare 1202 may exhibit an increasing diameter along its axis, but it may have an X-shaped cross section as opposed to a circular cross section. In an embodiment, proximal cap 208 may also have a cap portion 1204 extending away from a neck 1206 of flare 1202, and cap portion 1204 may be sized and configured to seal or block an opening in hollow body 202 and passage 306.

Referring to FIG. 12B, a proximal cap 208, distal cap 206 configuration having a snap 1208 retainer is shown in accordance with an embodiment of the invention. In an embodiment, cap portion 1204 may include a snap 1208 that tapers inward and is intended to be press fit over a snap detent 1210 formed in hollow body 202. Due to the press fit, snap 1208 is retained over snap detent 1210, but upon pulling cap portion 1204 away from hollow body 202, snap 1208 and snap detent 1210 deflect outward and inward, respectively, to disengage and expose passage 306 to the surrounding environment.

Referring to FIG. 12C, a proximal cap 208, distal cap 206 configuration having a plug 1212 retainer is shown in accordance with an embodiment of the invention. In an embodiment, inner surface 302 defining passage 306 may be substantially cylindrical. For example, hollow body 202 may be a thin-walled cylinder, or inner surface 302 may not conform to the shape of outer surface 204, e.g., outer surface 204 may be ellipsoidal and inner surface 302 may be cylindrical. Proximal cap 208 may include a plug 1212 to insert and be retained within passage 306. For example, plug 1212 may be sized to provide a press fit against inner surface 302. Alternatively, length and/or material of plug 1212 may be sufficient to generate friction against inner surface 302 that retains plug 1212. In an embodiment, proximal cap 208 may also have a cap portion 1204 extending away from a neck 1206 of plug 1212, and cap portion 1204 may be sized and configured to seal or block an opening in hollow body 202 and passage 306.

Referring to FIG. 12D, a proximal cap 208, distal cap 206 configuration having a hollow body 202 with an integrally formed cap is shown in accordance with an embodiment of the invention. In an embodiment, cap portion 1204 may include a cap wall 1214 that is contiguously formed with body wall 304 and shares a common outer surface 204. More specifically, cap wall 1214 may have a complementary shape, e.g., provide an end region of an ellipsoid shape, relative to body wall 304. Furthermore, cap wall 1214 may be thinner than body wall 304, making it more susceptible to piercing by cannulation member 1002.

In another embodiment, rather than continuing along the surface path defined by body wall 304, cap wall 1214 may change angles substantially. For example, while outer surface 204 may be ellipsoidal about a central axis, cap wall 1214 may occupy a plane that is perpendicular to that axis. Alternatively, rather than continuing to extend in the same direction as outer surface 204, cap wall 1214 may reverse directions, i.e., may be concave relative to outer surface 204 rather than convex as shown in FIG. 12D.

Referring to FIG. 12E, a proximal cap 208, distal cap 206 configuration having an integrally formed cap with puncture promoting features is shown in accordance with an embodiment of the invention. Cap portion 1204 may be weakened in one or more locations to locally promote puncture by cannulation member 1002. For example, cap portion 1204 may have one or more depression 1216 that creates local thinning of cap wall 1214 and therefore weakens cap wall 1214 to allow cannulation member 1002 to penetrate through cap portion 1204 more easily. Depression 1216 may be formed by drilling, stamping, ablating, or any other method that provides for localized thinning of material. Furthermore, depression 1216 may extend entirely through cap wall 1214, i.e., may be a perforation, and have a diameter that does not allow an ovum to pass. For example, a perforation may have a diameter less than about 0.1 mm.

Depressions or perforations may be formed in a pattern that facilitates predictable puncturing of cap portion 1204. For example, one or more depression 1216 may be formed in a crossing pattern with a center near a distal tip 1218 of cap portion 1204. Thus, upon puncturing of cap portion 1204 by cannulation member 1002, cap portion 1204 tears outward in a substantially uniform manner along the seams defined by the pattern. As a result, in an embodiment, an opening in cap portion 1204 that exposes passage 306 would have a maximum diameter centered about distal tip 1218, which may also to approximately coincide with a central axis of the fallopian tube.

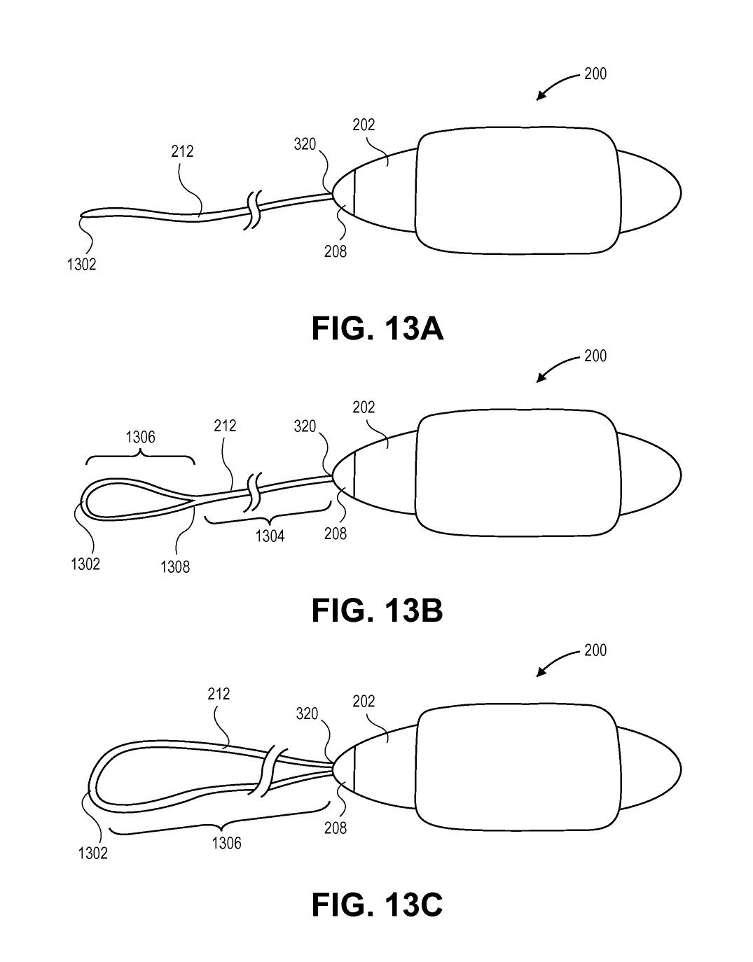

FIGS. 13A-13C provide various views of tail portions of an occlusion device in accordance with an embodiment of the invention. The various configurations are intended to be illustrative, and not exhaustive, of the numerous tail designs that may be used.

Referring to FIG. 13A, an occlusion device 200 having a tail 212 with a generally elongated shape is shown in accordance with an embodiment of the invention. Tail 212 may extend from proximal cap 208 to trail away from hollow body 202. More specifically, tail 212 may extend from a distal end 320 coupled with proximal cap 208 to a proximal end 1302. Tail 212 may have a generally elongated body between distal end 320 and proximal end 1302. For example, although tail 212 may be flexible and compliant, the elongated body of tail 212 may follow an arcuate path that does not cross back on itself. That is, tail 212 may be without a loop feature.

Referring to FIG. 13B, an occlusion device 200 having a tail 212 with a generally elongated body having a loop feature is shown in accordance with an embodiment of the invention. Tail 212 may extend from proximal cap 208 to trail away from hollow body 202. More specifically, tail 212 may extend from a distal end 320 coupled with proximal cap 208 to a proximal end 1302. In addition to having an elongated body portion 1304, tail 212 may also include a loop portion 1306. As in FIG. 13A, the elongated body portion may follow a path that is generally straight or arcuate but does not reverse on itself. However, loop portion 1306 may instead include an inflection point at proximal end 1302 in which tail 212 curves into a loop. Thus, loop portion 1306 may be contiguous with proximal end 1302, i.e., proximal end 1302 may be along the path of loop portion 1306. The loop may be secured at a securement point 1308, which forms a transition between elongated body portion 1304 and loop portion 1306. Securement point 1308 may be formed in several manners, including by tying, bonding, or otherwise securing tail 212 to itself at securement point 1308. Alternatively, elongated body portion 1304 and loop portion 1306 may be separately formed parts that are coupled with each other at securement point 1308.

Referring to FIG. 13C, an occlusion device 200 having a tail 212 with a generally elongated body having a loop feature is shown in accordance with an embodiment of the invention. Tail 212 may extend from proximal cap 208 to trail away from hollow body 202. More specifically, tail 212 may extend from a distal end 320 coupled with proximal cap 208 to a proximal end 1302. Tail 212 may include a loop portion 1306 between distal end 320 and proximal end 1302. As in FIG. 13B, the loop portion 1306 may reverse on itself at the inflection point contiguous with proximal end 1302. However, loop portion 1306 may extend over the entire length between proximal cap 208 and proximal end 1302. That is, both sides of the loop may meet and/or be coupled with proximal cap 208 rather than being joined at securement point 1308. More specifically, tail 212 may be coupled with proximal cap 208 at distal end 320 using any of the manners described above, including by stamping tail 212 ends to prevent loop portion 212 from being pulled out of proximal cap 208, or by adhesively bonding tail 212 inside of a bore within proximal cap 208.

Occlusion device 200 components may be formed using various well known manufacturing processes. For example, hollow body 202 may be formed using molding, e.g., injection molding, or casting processes. Alternatively, other components, such as tissue ingrowth fibers or expandable layer 210 may be formed by electrospinning onto a target having a desired shape. In addition to bulk formation of components using, e.g., casting, multiple components can be assembled and coupled together after formation. For example, expandable layer 210 may be formed separately from hollow body 202 and then bonded to hollow body 202 using a biocompatible adhesive, such as a biocompatible cyanoacrylate adhesive. Similarly, distal anchor 702 and/or proximal anchor 704 may be separately formed by casting, stamping, bending and heat treating, etc., before being attached to hollow body 202 at attached end 804. Attachment of attached end 804 to hollow body 202 may be made in numerous manners, including by adhesive or thermal weld, tying, press fitting, etc. For example, in an embodiment, shrink tubing may be used to wrap around and sandwich attached end 804 against hollow body 202.

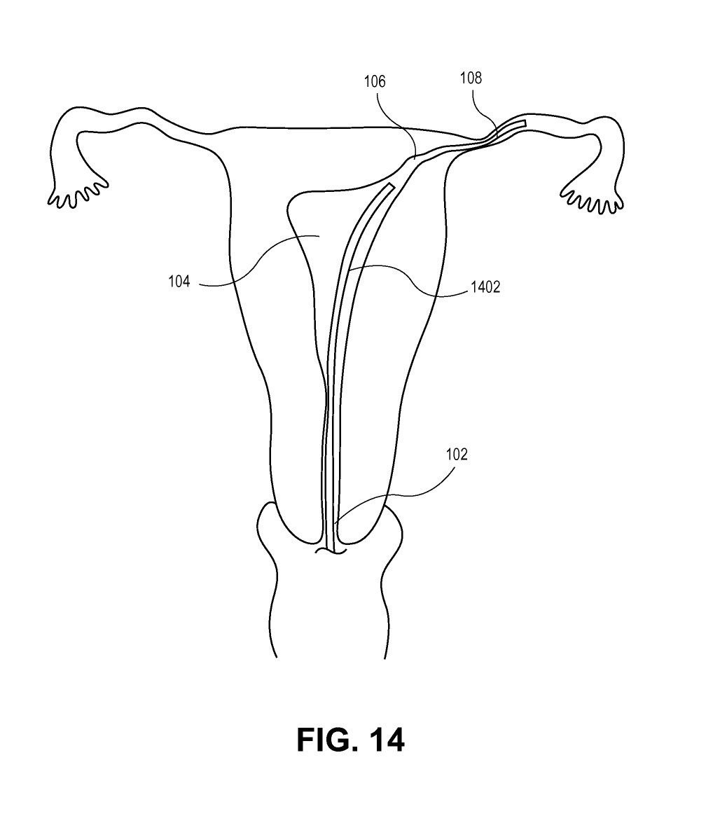

Referring to FIG. 14, an endoscope accessing an ostium of a fallopian tube is shown in accordance with an embodiment of the invention. An endoscope 1402 is introduced through cervix 102 into uterus 104 under optical direction. The physician directs the distal end of endoscope 1402 toward ostium 106 of the fallopian tube. Uterus 104 may be irrigated and/or distended. Once ostium 106 is located and endoscope 1402 is oriented toward ostium 106, a delivery system is advanced distally from endoscope 1402 into ostium 106.

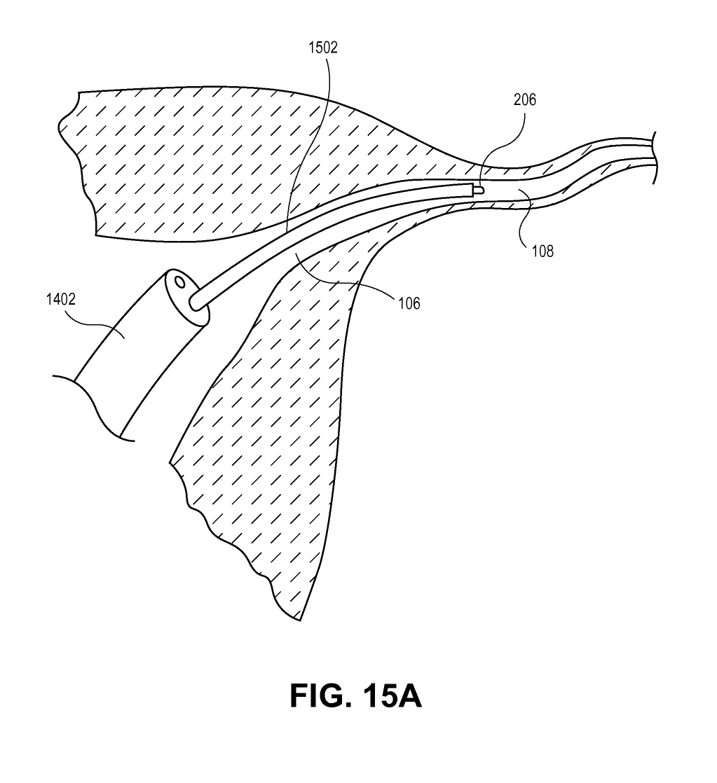

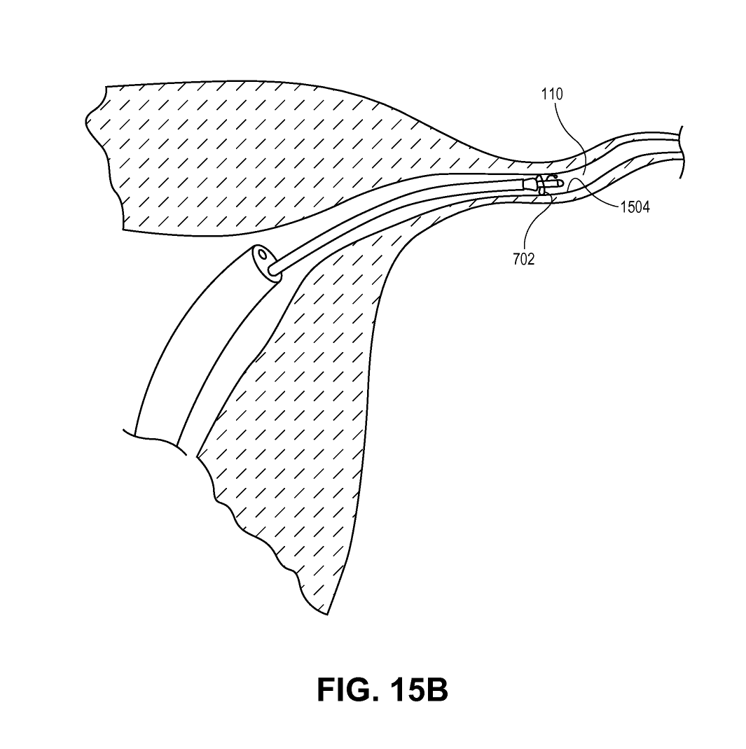

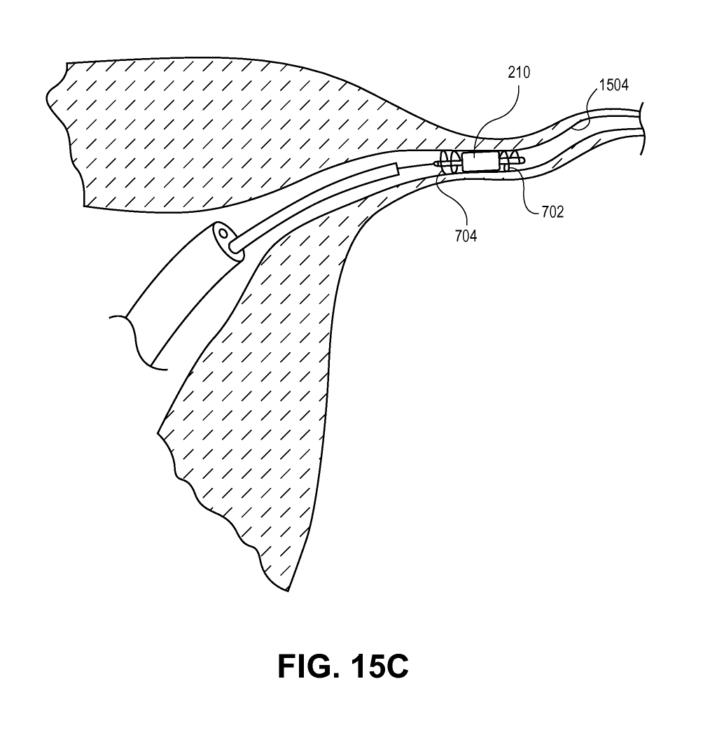

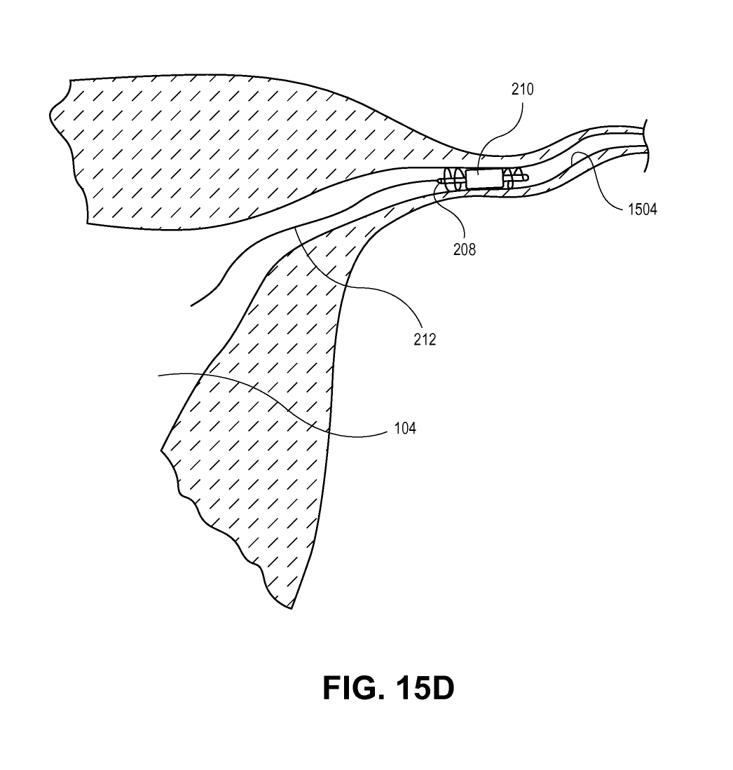

FIGS. 15A-15D show a delivery of an occlusion device to occlude a fallopian tube in accordance with an embodiment of the invention. Referring to FIG. 15A, in one embodiment, the delivery system includes a delivery catheter 1402 that encompasses at least a portion of occlusion device 200. For example, distal cap 206 may extend outward from delivery catheter 1402 to provide an atraumatic tip that promotes tracking, while proximal cap 208 may be retained within a lumen of delivery catheter 1402. Delivery catheter 1402 may be a microcatheter having a uniform inner diameter that conform to, and constrains, occlusion device 200 during delivery.

To further aid navigation, in an embodiment, passage 306 may be filled with a contrast agent to allow for visualization under different imaging modalities. For example, passage 306 may be filled with a radiopaque contrast agent, e.g., iodinated contrast medium, to facilitate imaging under fluoroscopy. Alternatively, passage 306 may be filled with a magnetic resonance signal enhancing substance, e.g., gadolinium, or an ultrasound scattering substance, e.g., microbubble contrast agent. The contrast agent-filled hollow body 202 promotes navigation and visualization of occlusion device 200 through the fallopian tube. Furthermore, the contrast agent may be biocompatible, given that contrast agent will leak into the fallopian tube following the opening of passage 306 to the surrounding environment.

In an embodiment, delivery catheter 1402 includes a visual marker which can be seen from the scope of a hysteroscope. The marker is preferably positioned partially within ostium 106 and partially within uterus 104 to indicate that occlusion device 200 is disposed at the target position. In an embodiment, the target position is within, or proximal to, isthmus segment 110. For example, the target position may be about 3-4 cm from ostium 106.

Referring to FIG. 15B, the positioned occlusion device 200 is deployed within the fallopian tube. In an embodiment, delivery catheter 1402 is withdrawn over occlusion device 200, causing hollow body 202 to become exposed to the surrounding environment and distal anchor 702 to expand outward into contact with the fallopian tube wall 1404. A pushing rod may be used within delivery catheter 1402 to press against a proximal end of occlusion device 200 while delivery catheter 1402 is withdrawn. Alternatively, the pushing rod may be used to push and deploy occlusion device 200 from delivery catheter 1402. Thus, distal anchor 702 resiliently expands through spring action or material phase transformation and secures occlusion device 200 to the fallopian tube. As delivery catheter 1402 is withdrawn further from occlusion device 200, expandable layer 210, e.g., hydrogel layer, begins to swell and to occlude the fallopian tube.

Referring to FIG. 15C, the hydrogel of expandable layer 210 may expand to fill areas of the fallopian tube to block the ovarian pathway. Furthermore, as delivery catheter 1402 is withdrawn even further, proximal anchor 704 may resiliently expand to contact and secure hollow member to the fallopian tube wall 1404. Thus, occlusion device 200 may become secured within fallopian tube by proximal anchor 704, distal anchor 702, and/or expandable layer 210.

Referring to FIG. 15D, further withdrawal of delivery catheter 1402 releases occlusion device 200 entirely, including tail 212 that trails proximally from proximal cap 208. In an embodiment, tail 212 extends limply from proximal cap 208 within, e.g., intramural portion 108 of the fallopian tube, into uterus 104. Furthermore, in the deployed state, the fallopian tube may be occluded by the expandable layer 210 as it swells outward, as well as by proximal cap 208 and distal cap 206 that enclose passage 306 and prevent an ovum from traveling therethrough. Thus, following deployment of occlusion device 200 into the fallopian tube, long-term contraception is achieved.

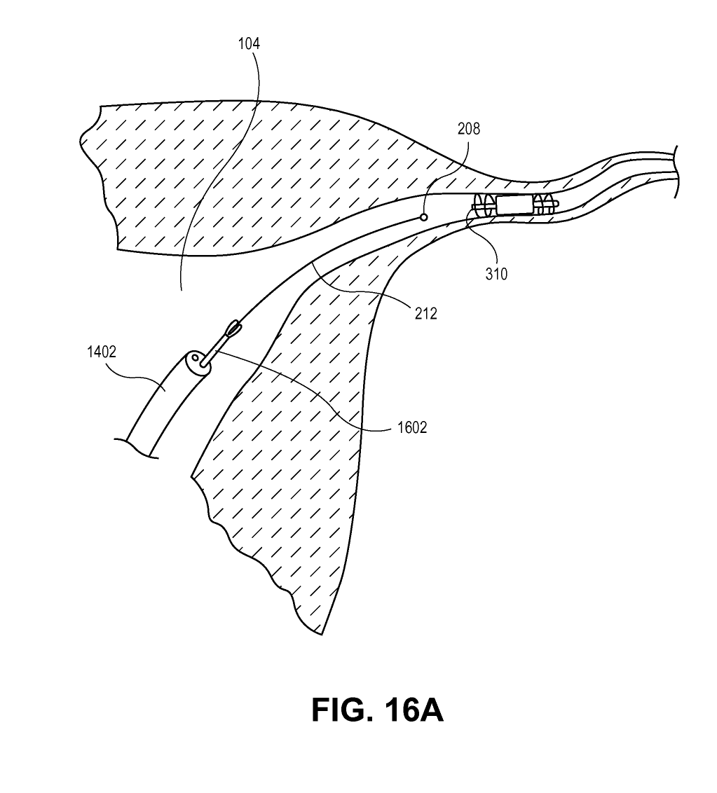

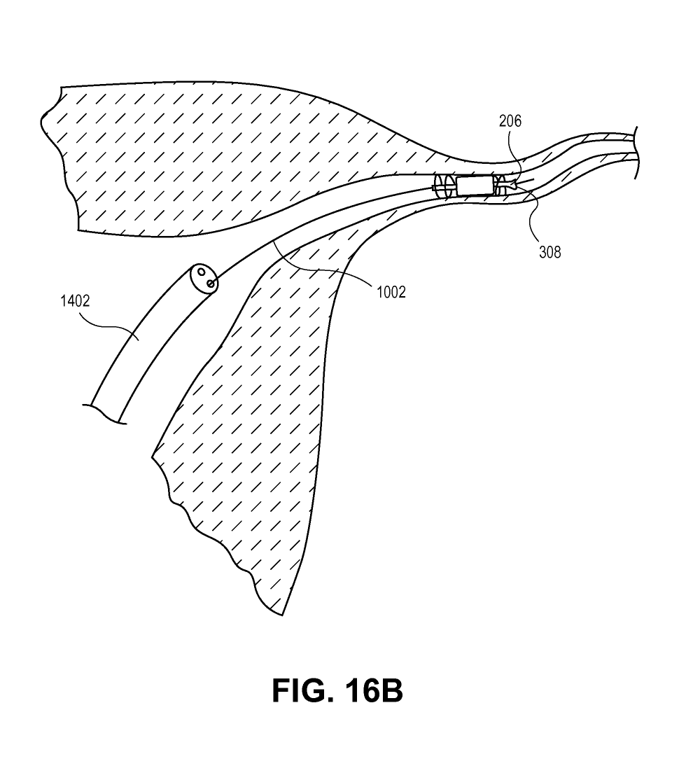

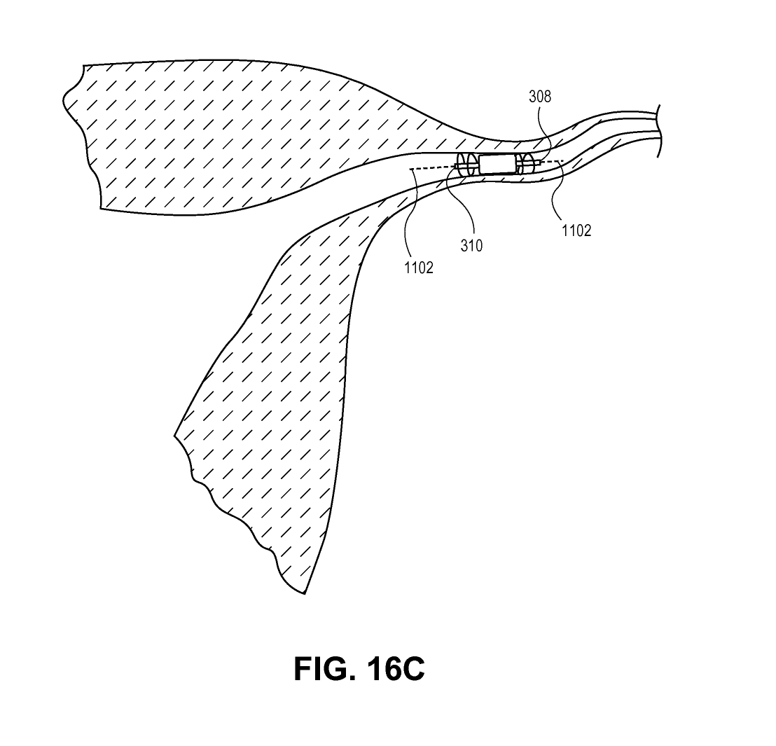

FIGS. 16A-16C show an unblocking of the fallopian tube to reverse contraception in accordance with an embodiment of the invention. Referring to FIG. 16A, tail 212 may act as a marker to be visualized by an endoscope 1402 inserted transcervically into uterus 104. For example, endoscope 1402 may be able to visualize a proximal tip of tail 212. Tail 212 may be visible as a result of its color, which may appear bright or contrast with uterus 104 tissue. Alternatively, tail 212 may be radiopaque to enable external visualization using non-optical imaging modalities, e.g., fluoroscopy. To enhance the radiopacity of tail 212, radiopaque fillers such as iodine and heavy metal compositions described above may be incorporated within tail 212 body. After identifying tail 212, a retrieval device 1602 may be extended from endoscope 1402 to grip tail 212. For example, retrieval device 1602 may include biopsy forceps to clamp tail 212 under vision provided by endoscope 1402.

After gripping tail 212, proximal cap 208 may be opened by pulling tail 212 to dislodge proximal cap 208 from hollow body 202 and retrieve proximal cap 208 through endoscope 1402. Following removal of proximal cap 208, proximal end 310 of passage 306 is openly exposed to the proximal region of the fallopian tube. In an embodiment as described above, removal of proximal cap 208 may also dislodge and retrieve distal cap 206 through passage 306 due to connector 602 between proximal cap 208 and distal cap 206.

In an alternative embodiment, retrieval device 1602 may include a hook or snare to capture a loop feature of tail 212, such as loop portion 1306 shown in FIGS. 13B-13C. Thus, rather than clamping tail 212, a hook may be placed through loop portion 1306 and pulled to grasp tail 212 at proximal end 1302. Sufficient force may be applied through the hook or snare to dislodge proximal cap 208 from hollow body 202 and retrieve proximal cap 208 through endoscope 1402.

Referring to FIG. 16B, in an embodiment in which proximal cap 208 and distal cap 206 are not coupled with each other, distal cap 206 may be opened by introducing cannulation member 1002 through endoscope 1402. Cannulation member 1002 may be a guidewire, cannula, or a catheter device having an outer diameter that fits through passage 306. In an embodiment, cannulation member 1002 may access passage 306 through a microcatheter. For example, a microcatheter may be introduced over tail 212 to a proximal end of occlusion device 200 before removing proximal cap 208. After removing proximal cap 208 by pulling it through the microcatheter, a guidewire may then be introduced through the microcatheter directly to the proximal end with minimal steering.

Following access, cannulation member 1002 may be advanced further through passage 306 to contact and pierce distal cap 206 and to expose distal end 308 of passage 306 to a distal region of the fallopian tube. As described above, cannulation of distal cap 206 may be performed in a multi-stage process, in which a guidewire is first advanced through distal cap 206 and then used as a rail to deliver a catheter through hollow body 202, which widens the opening in distal cap 206.

Referring to FIG. 16C, after opening both proximal cap 208 and distal cap 206, passage 306 is no longer enclosed and passage axis 1102 provides a pathway for an ovum to travel from the distal region of the fallopian tube to the proximal region of the fallopian tube. Accordingly, contraception is reversed to allow the patient to conceive.

In the foregoing specification, the invention has been described with reference to specific embodiments thereof. It will be evident that various modifications may be made to these embodiments without departing from the broader spirit and scope of the invention, as set forth in the following claims. The specification and drawings are, accordingly, to be regarded in an illustrative sense rather than a restrictive sense.

What is claimed is:

1. An occlusion device comprising:
a hollow body including an outer surface and a passage, the passage having a proximal end and a distal end;
a plurality of caps covering the proximal end and the distal end of the passage to enclose the passage within the hollow body; and
an expandable layer on the outer surface.

2. The occlusion device of claim 1, further comprising one or more anchors coupled with the outer surface, the one or more anchors having an expandable diameter.

3. The occlusion device of claim 1, further comprising a flexible tail coupled with a proximal cap of the plurality of caps, wherein the flexible tail trails proximally from the proximal cap.

4. The occlusion device of claim 3, wherein the flexible tail includes a radiopaque filler.

5. The occlusion device of claim 3, wherein the flexible tail includes an elongated body trailing between a distal end at the proximal cap and a proximal end.

6. The occlusion device of claim 5, wherein the elongated body fits within a microcatheter lumen to allow a microcatheter to be introduced over the flexible tail.

7. The occlusion device of claim 5, wherein the flexible tail includes a loop portion contiguous with the proximal end.

8. The occlusion device of claim 7, wherein the loop portion is further contiguous with the distal end.

9. The occlusion device of claim 1, further comprising a contrast agent filling the enclosed passage.

10. The occlusion device of claim 1, wherein the outer surface includes a shape selected from the group consisting of: convex, cylindrical, and concave.

11. The occlusion device of claim 10, wherein a length between the proximal end and the distal end is between 2 and 35 mm.

12. The occlusion device of claim 1, wherein one or more of the plurality of caps are integrally formed with the hollow body.

13. The occlusion device of claim 12, wherein the one or more integrally formed caps include a penetrable wall configured to be pierced by a cannulation member.

14. The occlusion device of claim 13, wherein the penetrable wall is thinner than an adjacent wall of the hollow body.

15. The occlusion device of claim 14, wherein the penetrable wall includes one or more depressions.

16. The occlusion device of claim 1, wherein one or more of the plurality of caps are formed separately from the hollow body, and wherein the separately formed caps plug the passage.

17. The occlusion device of claim 16, wherein the plurality of caps are coupled with each other by a connector.

18. The occlusion device of claim 1, wherein the expandable layer includes a non-slip surface.

19. The occlusion device of claim 18, wherein the expandable layer includes a hydrogel.

20. The occlusion device of claim 19, wherein the hydrogel is selected from the group consisting of biostable and bioresorbable hydrogels.

21. The occlusion device of claim 20, wherein the expandable layer includes a radiopaque filler in the hydrogel.

22. The occlusion device of claim 18, wherein the expandable layer includes a shape memory polymer.

23. The occlusion device of claim 22, wherein the shape memory polymer is selected from the group consisting of biostable and bioresorbable shape memory polymers.

24. The occlusion device of claim 2, wherein the one or more anchors include an expandable coil having an attached end coupled with the outer surface and an unattached end configured to expand radially apart from the attached end when the expandable coil is unconstrained.

25. A method comprising:
delivering an occlusion device into a fallopian tube, wherein the occlusion device includes a plurality of caps enclosing a passage within a hollow body, and an expandable layer on an outer surface of the hollow body;
expanding the expandable layer to occlude the fallopian tube; and
opening the plurality of caps to uncover the passage and unblock the fallopian tube.

26. The method of claim 25, further comprising expanding an anchor coupled with the outer surface into contact with the fallopian tube to secure the occlusion device to the fallopian tube.

27. The method of claim 25, wherein opening the plurality of caps comprises dislodging one or more of the caps.

28. The method of claim 27, further comprising introducing a microcatheter over a flexible tail coupled with a proximal cap of the plurality of caps, wherein the flexible tail trails proximally from the proximal cap.

29. The method of claim 25, wherein opening the plurality of caps comprises piercing one or more of the caps.
